(19) United States
(12) Patent Application Publication
(10) Pub. No.: US 2025/0269094 A1
(43) Pub. Date: Aug. 28, 2025

(54) CONTINUOUS FIBER SUPPORT BARRIER FOR ENGINEERED VASCULAR NETWORKS

(71) Applicant: IVIVA Medical, Inc., Medford, MA (US)

(72) Inventors: Charles C. Klassen, Jamaica Plain, MA (US); Andrew Ford, Nashua, NH (US); Manjot Singh, Woburn, MA (US)

(21) Appl. No.: 18/858,294

(22) PCT Filed: Apr. 20, 2023
(86) PCT No.: PCT/US2023/019321

§ 371 Date: Oct. 18, 2024

Related U.S. Application Data
(60) Provisional application No. 63/333,533, filed on Apr. 21, 2022.
(60) Provisional application No. 63/333,107, filed on Apr. 20, 2022.

(51) Int. Cl. A61L 31/04; A61L 31/14; A61L 31/16

(52) U.S. Cl. CPC A61L 31/048; A61L 31/042; A61L 31/044; A61L 31/045; A61L 31/046; A61L 31/148; A61L 31/16

(57) ABSTRACT

Disclosed herein are apparatuses comprising one or more channels defining a luminal space, wherein the luminal space is defined by an inner wall of fibrous basement membrane material, as well as methods of their manufacture and use.

### RELATED APPLICATIONS

This application claims the benefit of U.S. Provisional Application No. 63/333,107, filed Apr. 20, 2022, and U.S. Provisional Application No. 63/333,533, filed Apr. 21, 2022, the contents of both of which are hereby incorporated by reference in their entirety.

### BACKGROUND OF THE INVENTION

Bioengineering of tissues and organ grafts of human scale holds the promise of addressing donor organ shortage and providing novel tissues for treatment of conditions for which transplant is not readily available. However, realization of this goal likely requires recapitulation of native endothelial structures and specifically a continuous basement membrane.

Current methods of fabricating an engineered vascular network do not allow for creating a continuous basement membrane structure surrounding the channels. This structure is important for proper vascular function because it provides a stable substrate for the endothelial cell layer while also providing a substantial physical barrier to cell infiltration, aneurysm, and rupture from pressure. In the context of blood-contacting bioengineered grafts for extracorporeal perfusion or implantation via vascular anastomosis, a continuous engineered basement membrane structure could provide a means of modulating or inhibiting native immune system interaction with the engineered tissue graft.

There is a need to create an engineered vascular network with defined architecture that incorporates a continuous basement membrane structure inside a graft.

### SUMMARY OF THE INVENTION

Applicants have surprisingly discovered that by utilizing electrospinning and suitable sacrificial material, a vascular network surrounded by a continuous fibrous membrane can be fabricated. Such vascular network is highly advantageous, as it can withstand higher pressures than other engineered vascular networks, making it suitable for higher pressure in vivo applications. Further, such vascular network can be designed to reduce or eliminate immunogenicity of cells implanted inside the network. Moreover, such vascular networks enable use of lower density surrounding material which better mimics some body tissues (e.g., epithelial tissue, soft tissue) and, in some applications, can be susceptible to remodeling by the host.

Some aspects of the invention are directed to an apparatus comprising one or more channels defining a luminal space, wherein the luminal space is defined by an inner wall of fibrous basement membrane material. In some embodiments, the one or more channels form a vascular channel network. In some embodiments, the vascular channel network has a first end configured to be in fluid communication with a fluid supply and a second end configured to connect in fluid communication with a fluid outlet.

In some embodiments, the apparatus further comprises a scaffold material contacting an outer wall of the fibrous basement membrane material. In some embodiments, the scaffold material comprises one or more additional vascular channel networks that, optionally, are defined by an inner wall of fibrous basement membrane material. In some embodiments, the scaffold material comprises cells or bulk tissue. In some embodiments, the scaffold material has insufficient mechanical strength to define the luminal space in the absence of the fibrous basement membrane material support.

In some embodiments, the inner wall of fibrous basement membrane material inhibits or prevents an immunological reaction by a subject to cells and other substances in the luminal space when implanted in the subject.

In some embodiments, the fibrous basement membrane material comprises pores of sufficient size to enable diffusion of one or more biologically relevant molecules. In some embodiments, the one or more channels are capable of withstanding at least 60 mmHg internal pressure. In some embodiments, the vascular channel network is capable of withstanding at least 60 mmHg internal pressure.

In some embodiments, the fibrous basement membrane material comprises one or more of gelatin, gelatin composites, collagen, fibrin, chitosan, nitrocellulose, polylactic acid, polycaprolactone, polyethylene glycol, polyethylene glycol diacrylate or other biopolymers, polymers, or decellularized tissue or extra-cellular matrix that has been liquefied or homogenized.

In some embodiments, the fibrous basement membrane material comprises electrospun fibers. In some embodiments, the fibrous basement membrane material comprises electrospun fibers comprising a first component selected from the group consisting of polycaprolactone, polyethylene glycol, and polyethylene glycol diacrylate, and a second component selected from the group consisting of gelatin, collagen and fibrin, and wherein the fibrous basement membrane has a thickness of 0.5-30 micrometers.

In some embodiments, the fibrous basement membrane material has been subjected to one or more post-fabrication treatments selected from compression, annealing, chemical crosslinking, stretching, drawing, heat treatment, and solvent welding, whereby the treated fibrous basement membrane material is imparted enhanced mechanical properties compared to a fibrous basement membrane material not receiving one or more of the post-fabrication treatments. In some embodiments, the enhanced mechanical properties are selected from the group consisting of enhanced tensile strength, enhanced tensile modulus, enhanced abrasion resistance, enhanced thermal stability, enhanced elongation at break, enhanced hardness, enhanced crystallinity and combinations thereof. In some embodiments, the post-fabrication treatment comprises solvent welding. In some embodiments, the solvent welding is conducted in the presence of pressure imparted by opposing support substrates. In some embodiments, post-fabrication treatment comprises heat treatment in combination with pressure imparted by opposing support substrates.

In some embodiments, the apparatus is implanted in a subject. In some embodiments, the apparatus is extracorporeal to a subject.

In some embodiments, the luminal space comprises one or more cells selected from endothelial cells, epithelial cells, mesenchymal cells, induced pluripotent stem cell derived cells, endocrine cells, and stromal cells.

In some embodiments, the apparatus is configured to function as an artificial kidney, pancreas, lung, heart muscle, liver, spleen, small bowel, large bowel, neural tissue, skeletal muscle, composite tissue, fat tissue, bone tissue, or skin.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is an image of sacrificial material (water soluble polyvinyl alcohol (PVA)) fabricated into a hierarchical vascular pattern, surrounded by electrospun nanofibers of (PCL) and gelatin.

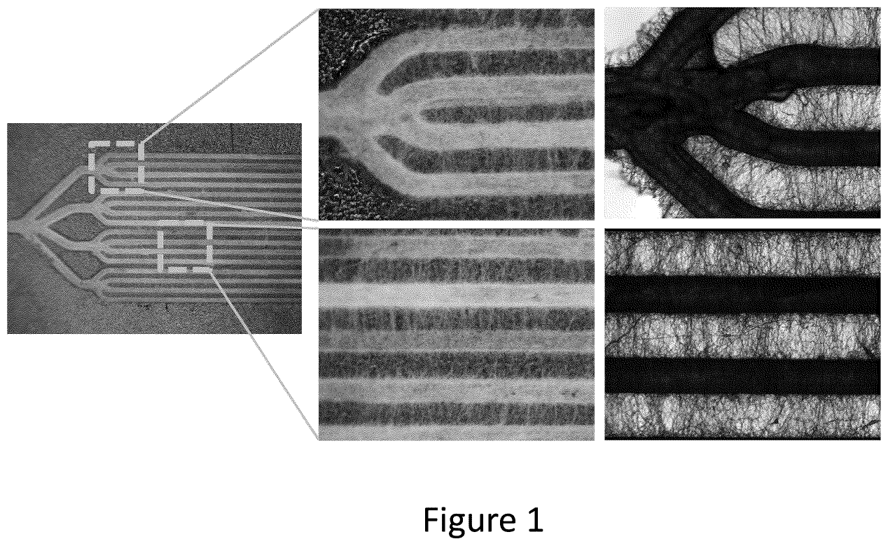

FIG. 2 is an SEM image of nanofiber membrane deposited around a printed Pluronic F127 vascular pattern (cross-section).

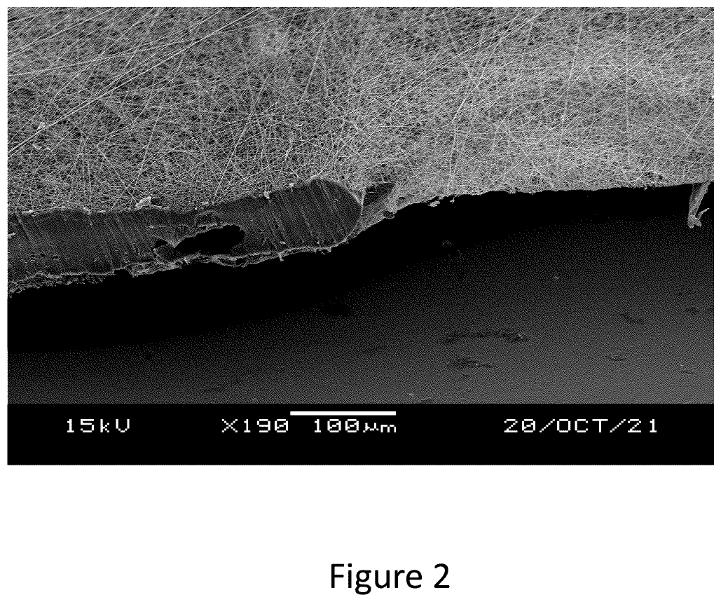

FIG. 3, left panel, is an image of printed sacrificial material being encased during electrospinning process. FIG. 3, right panel, is an image of a construct embedded into bulk gelatin material and perfused with dye.

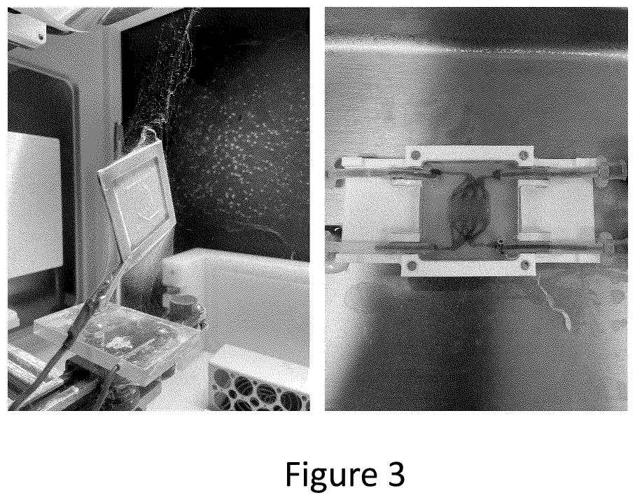

FIG. 4 is a graph showing burst pressure for a vascular network with or without fibrous support. N=3.

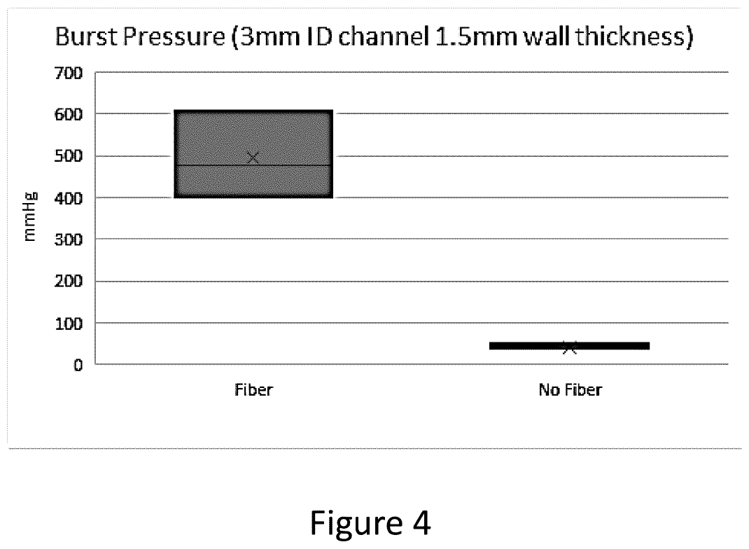

FIGS. 5a-d depict characteristics of electrospun fibrous membrane wrapping saturated with pure acetone and subjected to compression for a duration of 5 minutes. FIG. 5a depicts a scaffold having been formed by compression of an acetone saturated fibrous membrane upon a sacrificial substrate, wherein the compression lasted for a duration of 5 minutes. FIG. 5b is a SEM image of the fibrous membrane cross section after treatment. FIG. 5c is a SEM image of the fibrous membrane surface after treatment. FIG. 5d depicts the same scaffold with channels perfused with dye after treatment.

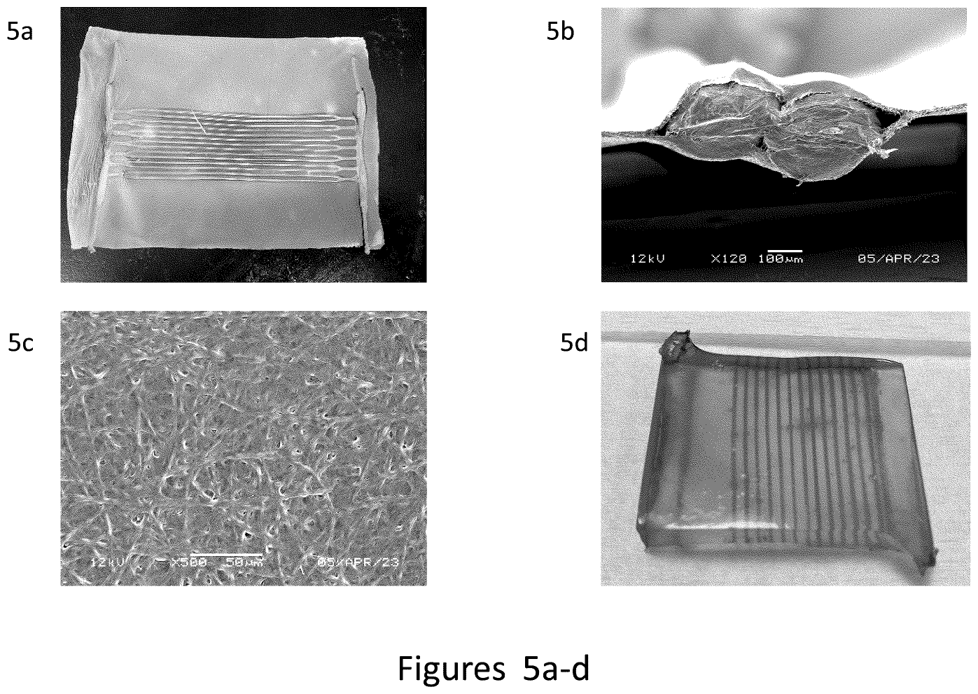

FIGS. 6a-d depict characteristics of a fibrous membrane wrapping saturated with pure acetone and subjected to compression for a duration of 1 minute. FIG. 6a depicts a scaffold having been formed by compression of an acetone saturated fibrous membrane upon a sacrificial substrate, wherein the compression comprises a duration of 5 minutes. FIG. 6b is a SEM image of the fibrous membrane cross section after treatment. FIG. 6c is a SEM image of the fibrous membrane surface after treatment. FIG. 6d depicts the same scaffold with channels perfused with dye after treatment.

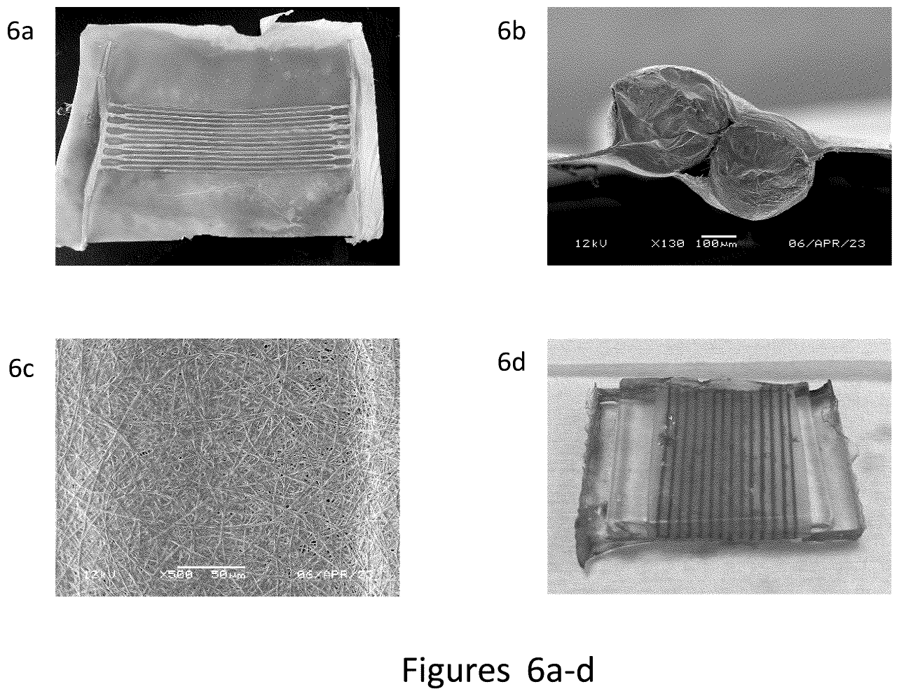

FIGS. 7a-d depict characteristics of a fibrous membrane wrapping sprayed with a small volume of pure acetone and subjected to compression for a duration of 5 minutes. FIG. 7a depicts a scaffold having been formed by compression of sacrificial substrate overlayed by a fibrous membrane sprayed with a small volume of pure acetone, wherein the compression comprises a duration of 5 minutes. FIG. 7b is a SEM image of the fibrous membrane cross section after treatment. FIG. 7c is a SEM image of the fibrous membrane surface after treatment. FIG. 7d depicts the same scaffold with channels perfused with dye after treatment.

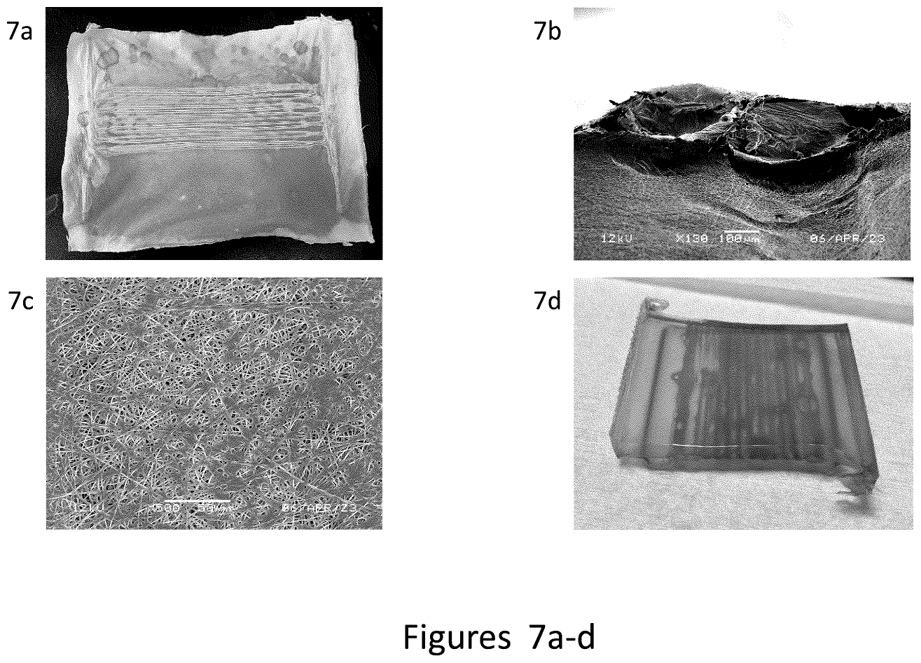

FIGS. 8a-d depict sequential assembly of a large scale tissue scaffold, using water soluble polyurethane as sacrificial material. FIG. 8a depicts a large scale tissue scaffold comprising a membrane having a water soluble polyurethane channel pattern printed thereon, undergoing close proximity electrospinning for membrane wrapping. FIG. 8b depicts electrospun fibers accumulating over the printed water soluble polyurethane pattern, wherein the location of fiber accumulation is influenced by collector electrode. FIGS. 8c and 8d are SEM images of cross section of the membrane and water soluble polyurethane pattern after close proximity electrospinning, at x160 and x120 magnification, respectively.

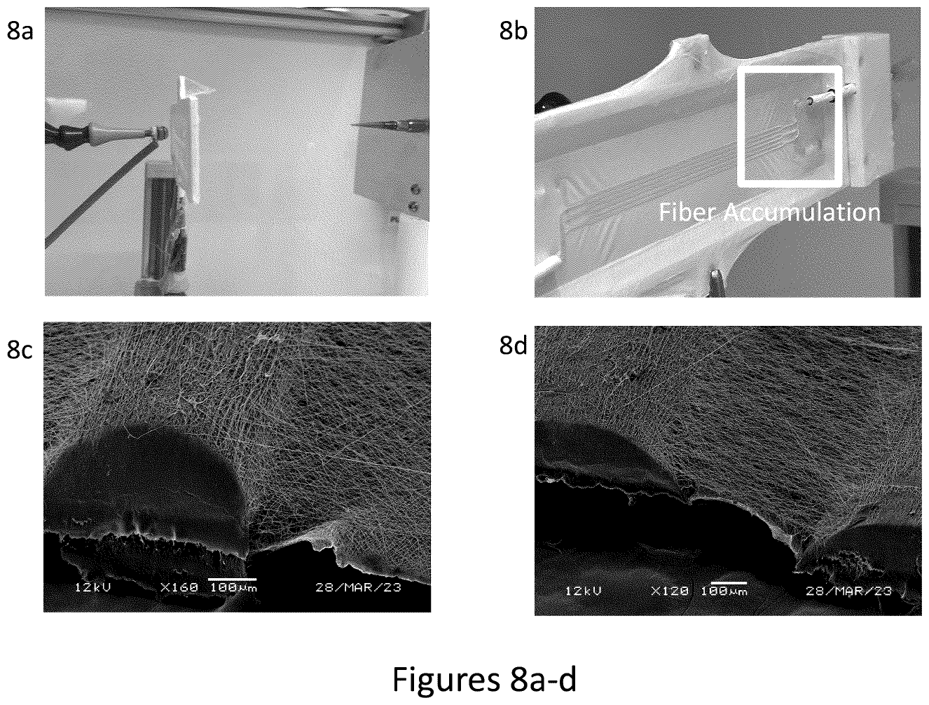

FIGS. 9a-b depicts characteristics of membrane wrapping after having undergone light compression of approximately 3-8 psi in addition to heat treatment comprising heating to approximately 55° C. for 18 hours. FIG. 9a is a SEM image taken at x55 magnification of the cross section of heat treated electrospun membrane surrounding water soluble polyurethane patterns. FIG. 9b is a SEM image taken at x180 magnification of the cross section of heat treated electrospun membrane surrounding water soluble polyurethane patterns.

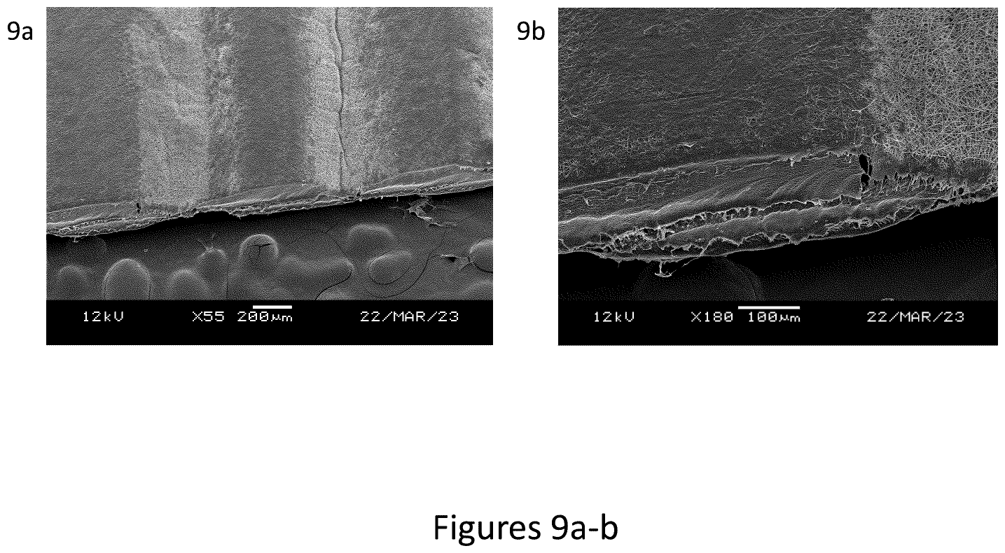

### DETAILED DESCRIPTION OF THE INVENTION

Provided herein are devices and compositions that provide a continuous barrier layer of controlled composition between vascular networks and engineered tissue material or grafts. Some embodiments are directed toward an engineered vascular network fabricated from sacrificial material, which serves as a substrate for fabrication of a basement membrane layer that is continuous and surrounds, e.g., completely, the vascular network, and provides separation from any additional tissue material placed outside of the vascular lumen. This membrane is fibrous and porous and mechanically robust, and it allows for movement of fluid and molecules of a certain size across the membrane, while also excluding molecules, particles, cells, and other material larger than a desirable size from passing through.

Applicants have surprisingly discovered that by utilizing electrospinning and suitable sacrificial material, a vascular network surrounded by a continuous fibrous membrane can be fabricated. Such vascular network is highly advantageous, as it can withstand higher pressures than other engineered vascular networks, making it suitable for higher pressure in vivo applications. Further, such vascular network can be designed to reduce or eliminate immunogenicity of cells implanted (or migrated) inside the network. Also, such vascular networks enable use of lower density surrounding material to better mimic some body tissues (e.g., epithelial tissue, soft tissue) and, in some applications, be suspectable to remodeling by the host.

Some aspects of the invention are directed to a device comprising a tissue scaffold that contains a vascular network surrounded by a continuous biocompatible fibrous basement membrane layer. In some embodiments, the continuous biocompatible fibrous basement membrane layer provides a complete barrier that prevents cells within the vascular network (e.g., blood cells) from contacting cells outside the network.

Another aspect of the invention is directed to an apparatus having one or more channels defining a luminal space. The luminal space may be defined by an inner wall of fibrous basement membrane material. A cross-sectional profile and a longitudinal profile of the one or more channels can encompass a variety of shapes and configurations. In one embodiment, the cross-sectional profile of the one or more channels has a cylindrical, approximately cylindrical or a partially flattened cylindrical shape. The longitudinal profile of the one or more channels includes linear, non-linear, or a combination of linear and non-linear configurations and adapts a bifurcated, non-bifurcated, or a networked configuration. In a preferred embodiment, the longitudinal profile of the one or more channels is non-linear and comprises a curved configuration, such as an S-curve configuration. Additionally, the cross-sectional profile of the one or more channels defines a diameter between about 6 μm and 10 cm, more preferably between about 15 μm and 5 cm and most preferably between about 60 μm and 1.5 cm. This cross-sectional diameter of the one or more channels can remain constant or vary over part or all of the longitudinal axis of the one or more channels.

In a preferred embodiment, the combination of the cross-sectional and longitudinal profiles results in the one or more channels configured to simulate lumens found naturally in a mammal. For example, the one or more channels are configured to simulate a substructure or combination of substructures of a mammalian cardiovasculature. Thus, in one embodiment the one or more channels may be configured to simulate the aorta, arteries, arterioles, arteriovenous anastomosis, capillaries, metarterioles, capillary beds, venules, veins or combinations thereof. In a particularly preferred embodiment, the one or more channels comprise at least two channels which are configured to form a vascular channel network.

In some embodiments, the vascular channel network has a first end configured to be in fluid communication with a fluid supply and a second end configured to connect in fluid communication with a fluid outlet. In such a configuration, the vascular channel network is utilized ex vivo or extracorporeally in a laboratory, hospital or other research and clinical settings or is used in vivo and is directly implanted into a subject. When the vascular channel network is configured for use ex vivo or extracorporeally, the fluid supply, the fluid outlets or both the fluid supply and fluid outlets comprise or are in fluid communication with one or more pumps, which initiate or sustain movement of a fluid within the vascular channel network. By way of non-limiting example, the pumps used in the invention are pumps commonly used in a laboratory or clinical setting including piston pumps, diaphragm pumps, peristaltic pumps, syringe pumps, pneumatic pumps, microfluidic pumps, infusion pumps, vacuum pumps and combinations thereof. Further, the fluid supply, the fluid outlets or both the fluid supply and the fluid outlets comprise or are in fluid communication with one or more valves for regulating the flow of a fluid within the vascular channel network. By way of non-limiting example, valves encompassed by this invention are those commonly used in the laboratory or clinical setting and include isolation valves, fluid control valves, metering valves, check valves and special purpose valves, wherein each of these valves may be classified as a gate valve, ball valve, pinch valve, diaphragm valve, needle valve, butterfly valve, plug valve or other type of valve. Optionally the invention may include one or more sensors for determining flow rate, temperature, pressure, etc.

In some embodiments, the apparatus has a scaffold material contacting an outer wall of the fibrous basement membrane material. In some embodiments, the scaffold material contains one or more additional vascular channel networks that, optionally, are defined by an inner wall of fibrous basement membrane material. In some embodiments, the scaffold material may contain multiple layers of material, each of which is solid, semi-solid or defines one or more channels or channel networks therein. In some embodiments, at least one channel or channel network of at least one scaffold layer interfaces with at least one other channel or channel network of at least one other scaffold layer or a channel defined by the outer wall of the fibrous basement membrane material. In a preferred embodiment, the interface between channels of different scaffold layers is via direct fluid communication or is an interface through a fibrous membrane.

In some embodiments, the scaffold material comprises cells or bulk tissue. The types of cells and bulk tissue seeded and placed on or within the scaffold are not particularly limited and include any of the cells or bulk tissue types recited herein. In a preferred embodiment, the cells include stem cells, endothelial cells, cardiomyocytes, parietal epithelial cells, hepatocytes, biliary cholangiocytes, stellate cells, adipocytes, osteoblasts, osteocytes, osteoclasts, enterocytes, Goblet cells, enteroendocrine cells, Paneth cells, microfold cells, cup cells, tuft cells, or other cells which are present in the kidney, pancreas, lung, heart muscle, liver, spleen, small bowel, large bowel, neural tissue, skeletal muscle, composite tissue, fat tissue, bone tissue, or skin.

In some embodiments, the scaffold material has insufficient mechanical strength to define the luminal space in the absence of the fibrous basement membrane material support.

In some embodiments, the inner wall of fibrous basement membrane material inhibits or prevents an immunological reaction by a subject to cells and other substances in the luminal space when implanted in the subject.

In some embodiments, the fibrous basement membrane material comprises pores of sufficient size to enable diffusion of one or more biologically relevant molecules. The pore size of the pores in the nanofiber membrane may be any suitable size and is not limited. In one embodiment, the average or median pore size diameter is about 0.05 to about 0.6 μm. In another embodiment, the average or median pore size diameter is about 0.05 μm, 0.1 μm, 0.15 μm, 0.2 μm, 0.25 μm, 0.3 μm, 0.35 μm, 0.4 μm, 0.45 μm, 0.5 μm, 0.55 μm or about 0.6 μm. The porosity of the nanofiber membrane (Pnm), obtained by dividing volume of voids (Vv) by the total volume of nanofiber membrane measured (VTvm) (P=Vv/VTvm * 100%), may be any suitable porosity and is not limited. In some embodiments the porosity is at least 5%, 10%, 20%, 30%, 40%, 50%, 60%, 70%, 80%, 90% or more. In other embodiments the porosity is between 20-80%, 30-70% or 40-60%.

In some embodiments, the one or more channels are capable of withstanding at least 60 mmHg internal pressure. In another embodiment, the one or more channels are capable of withstanding an internal pressure of at least 80 mmHg, 100 mmHg, 150 mmHg, 200 mmHg, 250 mmHg, 300 mmHg, 350 mmHg, 400 mmHg, 450 mmHg, 500 mmHg, 550 mmHg, or at least 600 mmHg.

In some embodiments, the vascular channel network is capable of withstanding at least 60 mmHg internal pressure. In another embodiment, the vascular channel network is capable of withstanding an internal pressure of at least 80 mmHg, 100 mmHg, 150 mmHg, 200 mmHg, 250 mmHg, 300 mmHg, 350 mmHg, 400 mmHg, 450 mmHg, 500 mmHg, 550 mmHg, or at least 600 mmHg.

In some embodiments, the fibrous basement membrane material comprises one or more of gelatin, gelatin composites, collagen, fibrin, chitosan, nitrocellulose, polylactic acid, polycaprolactone, polyethylene glycol, polyethylene glycol diacrylate, biopolymers, polymers, or decellularized tissue or extra-cellular matrix that has been liquefied or homogenized.

In some embodiments, the fibrous basement membrane material comprises electrospun fibers formed from a binary, ternary, quaternary or quinary mixture of materials. Particularly preferred mixtures include a binary mixture of collagen and polycaprolactone. In one particularly preferred embodiment the collagen takes the form of bovine, porcine or fish gelatin with a molecular weight of 15-400 kDa. In some embodiments, the gelatin will have a bloom value of 30-300, a bloom value of 40-100, a bloom value of 100-200 or a bloom value of 200-280. Additionally, the gelatin is cross-linked. In another preferred embodiment, the polycaprolactone utilized to form electrospun fibers in the fibrous basement membrane has a molecular weight of 10-100 kDa, 25-80 kDa or 30-60 kDa. The individual materials utilized in the binary mixture are present in a ratio range of 1:10 to 10:1, preferably 1:4 to 4:1, or more preferably 1:2 to 2:1. In a particularly preferred embodiment, the individual materials in the binary mixture are present in a 1:1 ratio.

The thickness of the fibrous basement membrane is not particularly limited and will vary based upon a variety of factors, including the end use of the apparatus. In some embodiments, the fibrous basement membrane has a thickness of 0.5-30 μm, has a thickness of 3-25 μm or a thickness of 10-20 μm. When the fibrous basement membrane is designed for use under high pressures, the fibrous basement membrane will be configured with an increased thickness and will have a thickness of 10-30 μm. Conversely, when the fibrous basement membrane is configured for use under low or medium pressures or if increased elasticity of the membrane is desired, the fibrous basement membrane is configured with a lower thickness and will have a thickness of 0.5-20 μm.

In some embodiments, the apparatus is implanted in a subject. In some embodiments, the apparatus is configured to facilitate surgical anastomosis to a native tissue which defines an aperture or a luminal space. In a preferred embodiment, the apparatus is attached in fluid communication with an artery, a vein, a renal collecting duct, a bronchiole, an endocrine duct, a small intestine, or a large intestine. In another embodiment, the implanted apparatus functions as an artificial organ.

In some embodiments, the apparatus is extracorporeal to a subject. When the apparatus is configured for extracorporeal use, the apparatus is directly or indirectly attached and in fluid communication with a native tissue in the subject that defines an aperture or a luminal space. In another embodiment, the apparatus is supported or held by an exterior surface of the subject's body. In yet another embodiment, the apparatus is free-standing and rests on a level surface or is otherwise fixedly or removably attached to an external support near a subject. In another embodiment, the apparatus functions as an external artificial organ.

In some embodiments, the luminal space has one or more cells. These cells are not particularly limited and can include any of the cells described herein. In one embodiment, the one or more cells are endothelial cells, epithelial cells, mesenchymal cells, induced pluripotent stem cell derived cells, endocrine cells, or stromal cells. In a preferred embodiment, the luminal space comprises 2 different cells, 3 different cells, 4 different cells, or 5 or more different cells.

In some embodiments, the apparatus is configured to function as an artificial mammalian organ. In a preferred embodiment, the apparatus is configured to function as an artificial kidney, pancreas, lung, heart muscle, liver, spleen, small bowel, large bowel, neural tissue, skeletal muscle, composite tissue, fat tissue, bone tissue, or skin.

In some embodiments, this biocompatible fibrous basement membrane layer serves as a boundary between the endothelial region of the tissue scaffold and other regions, which may have lumen and channel networks of their own (kidney, lung, etc.) or which may be simple bulk tissue (muscle, etc.). This layer also provides mechanical support to the vascular network and prevents undesirable events such as aneurism or rupture due to excessive pressure, which are a major challenge for engineered vascular networks. In some embodiments, in addition to the mechanical support provided the biocompatible fibrous basement membrane layer, the layer also provides a controlled interface between the vascular fluid and surrounding tissue. Thus, in some embodiments, by controlling porosity and composition of the biocompatible fibrous basement membrane layer, it is possible to modulate or prevent undesirable interaction or effects such as immune response. This is in essence an encapsulation strategy for an engineered vascular network, providing a barrier between host and graft tissue at the vascular basement membrane.

In some embodiments, a hypoimmunogenic endothelium is provided inside of a tissue scaffold and the remaining tissue in the scaffold is isolated from the immune system via the biocompatible fibrous basement membrane layer, enabling a hypoimmune tissue graft or extracorporeal device.

Some aspects of the present disclosure are directed to fabrication of a single continuous membrane or plurality of fibrous membranes surrounding a sacrificial material in the form of the vascular channel network. These membranes are made from fibers, preferably nanofibers, which are fabricated through electrospinning. Electrospinning is known to a person of art. See, Xue et al., Chem. Rev. 2019, 119, 8, 5298-5415, and Teo et al., Nanotechnology 17 (2006) R89-R106; both incorporated herein by reference. The fibers are fabricated in such a way that they create a porous fiber membrane along the entirety of the sacrificial material defining the vascular channel network, preferable from direct electrospinning onto the sacrificial material, alternatively through fabrication and a subsequent deposition or encasing step.

In some embodiments, the electrospinning is carried out in close-proximity to the substrate/collector plate to be coated with electrospun fibers. In some embodiments the distance between tip and collector plate is less than 15 cm, less than 12 cm, less than 10 cm, less than 9 cm, less than 8 cm, less than 7 cm, less than 6 cm, less than 5 cm, less than 4 cm, less than 3 cm, or less than 2 cm. In some embodiments, the diameter of the inner aperture of the tip, the feed rate of the fiber precursor solution or melt, and/or the concentration of the polymer in the precursor solute or melt is reduced to produce a smaller diameter fiber. Additionally, the applied voltage may be adjusted to reduce the diameter of the formed fiber, such as by causing increased stretching or drawing of the ejected precursor solution or melt before deposition on the collector substrate. In some embodiments, volatility of any solvent for use in dissolving the fiber precursor material is carefully considered, along with other spinning parameters, to ensure proper fiber formation and deposition is achieved in the finished fibrous membrane. In certain embodiments, reducing the diameter of the fiber permits the evaporation of the solvent in the precursor solution, or cooling and hardening of the precursor melt, before arriving on the sacrificial substrate/collector plate as solid fiber. This can be especially important when distance between tip and sacrificial substrate/collector plate is reduced or small, as required in certain embodiments of the invention. In alternative embodiments, the feed rate, aperture size of the needle and applied voltage is selected to provide semi-solid fibers which anneal to each other upon deposition on the substrate/collector plate, thus reducing the duration of a post-fabrication treatment step, or even permitting the elimination of post-fabrication treatment altogether.

In some embodiments, the fiber diameter is less than 10 μm, less than 9 μm, less than 8 μm, less than 7 μm, less than 6 μm, less than 5 μm, less than 4 μm, less than 3 μm, less than 2 μm, or less than 1 μm. In some embodiments, a majority of fibers present in the fibrous membrane are nanofibers, with a diameter of 950 nm or less, 800 nm or less, 600 nm or less, 450 nm or less, or 200 nm or less. In some embodiments, the majority of fibers in the fibrous membrane are nanofibers having a diameter between approximately 100 nm and 750 nm, between approximately 100 nm and 500 nm, or between approximately 250 nm and 800 nm. In some embodiments, the fibrous membrane comprises fibers with a diameter of greater than 950 nm, greater than 2 μm, greater than 3 μm, greater than 4 μm, greater than 5 μm or more. In some embodiments, the diameter of the electrospun fiber is at least 5 μm, at least 6 μm, at least 7 μm, at least 8 μm, at least 9 μm or more.

In some embodiments, manufacture of the apparatuses disclosed herein does not involve forming a porous fibrous membrane directly on a sacrificial substrate, either through electrospinning or through any other means. Rather, in some embodiments, the porous fibrous membrane is formed and subsequently manipulated alone, or in combination with other fibrous membranes, to form one or more channels defining a luminal space. In some embodiments, one or more porous fibrous membranes are manipulated to form a vascular channel network, by contacting the one or more fibrous membranes with one or more channel templates. In preferred embodiments, this contacting is done under conditions suitable for inducing permanent morphological changes in the one or more porous membranes that mimics the shape of the one or more templates. Conditions suitable to induce permanent morphological changes in the one or more porous fibrous membranes include performing contacting in the presence of one or more of: elevated temperature, compression force, and one or more solvents. In some embodiments, the condition comprises application of compression force sufficient to stretch or draw the fibrous membrane around the one or more templates. In preferred embodiments, at least two conditions are present simultaneously to induce the permanent morphological changes in the one or more porous fibrous membranes upon contacting the one or more templates. In preferred embodiments, the one or more templates comprise a sacrificial substrate deposited directly onto a fibrous membrane. In some embodiments the sacrificial substrate is deposited onto a substrate which is suitable for releasing the sacrificial substrate once the porous fibrous membrane has been contacted with or applied thereto.

In some embodiments, initial fabrication of the electrospun fibrous membrane is followed by post-fabrication treatment. In some embodiments, the post-fabrication treatment includes, but is not limited to, one or more of compression, annealing, chemical crosslinking, stretching/drawing, and solvent welding.

In some embodiments, the post-fabrication treatment may be carried out between 20-22° C., or at a temperature greater than 22° C. In some embodiments, the post-fabrication step is performed below the glass transition temperature (Tg) of one, two or all of the materials used to form the fibrous membrane, such as at least 5° C., 10° C., 15° C., 20° C., 30° C. or more below the glass transition temperature.

In certain embodiments, the post-fabrication step is annealing performed at a temperature that is approximately equal to the glass transition temperature of the material used to form the fibrous membrane. For purposes of this application, approximately equal to the glass transition temperature (Tg) is defined as a published glass transition temperature of the material ±5° C., or ±5% of the published Tg, whichever provides the smaller temperature range. In some embodiments, the post-fabrication step is conducted at a temperature that falls between the glass transition temperature and the melting temperature of the material used to form the fibrous membrane. It will be appreciated by one of ordinary skill in the art that as the temperature is increased above the glass transition temperature, care should be taken to select an appropriate post-fabrication treatment time to ensure that porosity of the membrane is not destroyed through substantial melting of the fibrous membrane.

In some embodiments, the fibrous membrane is subjected to a post-fabrication step at a temperature of greater than 25° C., 30° C., 35° C., 40° C., 45° C., 50° C., 55° C., 60° C., 65° C., 70° C., 75° C., 80° C., 85° C., 90° C., 100° C., 125° C., 150° C., 160° C., 170° C., 180° C. or more. In some embodiments the post-fabrication step is carried out for a period of time of 1 second, 5 seconds, 10 seconds, 15 seconds, 30 seconds, 45 second, 1 minute, 2 minutes, 3 minutes, 4 minutes, 5 minutes, 6 minutes, 7 minutes, 8 minutes, 9 minutes, 10 minutes, 20 minutes, 30 minutes, 45 minutes, or 1 hour. In certain embodiments it is desired to conduct the post-fabrication treatment for a longer period of time, including a time of 1.5 hours, 2 hours, 3, hours, 4, hours, 5 hours, 6 hours, 7 hours, 8 hours, 9 hours, 10 hours, 15 hours, 16 hours, 17 hours, 18 hours, 19 hours, 20 hours, 25 hours, 1 day, 2 days, 3 days, or more.

In some embodiments the post-fabrication annealing step includes application of compression to facilitate wrapping of a sacrificial substrate with a fibrous membrane, as described herein. In some embodiments, application of compression at elevated temperature facilitates superior or more rapid bonding or annealing of the fibers. In some embodiments the post-fabrication annealing and compression steps are conducted at a temperature between 120-180° C., 40-70° C., 45-65° C., 50-60° C. or 53-57° C., and for a time period of 11-25 hours, 13-22 hours, 15-20 hours, or 17-19 hours. In certain preferred embodiments the compression and annealing steps are conducted for approximately 18 hours at approximately 55° C.

The magnitude of pressure utilized in the compression step is not particularly limited, as long as it is sufficient to facilitate the bonding or annealing of the fibers where they intersect, while protecting the fibrous membrane or substrate to which it is applied from damage. In some embodiments, the compression is applied by placing the fibrous membrane, and substrate upon which it was fabricated, between two opposing releasing substrates of a size, shape and hardness that permits application of sufficient force to induce bonding or welding of the fibers, without damaging the fibrous membrane or substrate to which it is applied. In some embodiments, the releasing substrates are soft silicone pads. In certain preferred embodiments, the compression step is carried out by placing fibrous membrane and sacrificial substrate between two silicon pads resting on a solid surface, wherein the only compression is generated by the weight of the silicon pad placed on top thereof.

In some embodiments, additional compression force may be applied by using a pressure applied by placing increasingly heavier objects on top of the upper silicon pad. In some embodiments, the pressure applied to the fibrous membrane and sacrificial substrate is in the compression treatment is less than 25 psi, less than 20 psi, less than 18 psi, less than 15 psi, less than 12 psi, less than 10 psi, less than 8 psi, less than 7 psi, less than 6 psi, less than 5 psi, less than 4 psi, less than 3 psi, less than 2 psi, less than 1 psi, less than 0.5 psi, less than 0.4 psi, less than 3 psi, less than 2 psi, or less than 0.1 psi. In some preferred embodiments, when the pressure is applied in presence of heat, the pressure is 0.05 psi to 2 psi, 0.05 psi to 1 psi or 0.1 to 0.8 psi. In some further embodiments, the pressure applied during the compression treatment is 0.5-5 psi, 2-8 psi, 5-14 psi, 3-7 psi, or 18-25 psi.

In some embodiments, the post-fabrication treatment comprises exposing the fibrous membrane to a solvent to facilitate welding together of partially softened or swollen fibers at their point of intersection. The solvent used for solvent welding should be capable of at least partially softening or swelling the fibers of the fibrous membrane material to facilitate welding of the fibers together, given a reasonably amount of time. In some embodiments, the solvent is non-toxic. In other embodiments, the solvent has a Hildebrand solubility parameter similar to that of the material used to form the electrospun fibrous membrane. In circumstances where a given concentrated solvent may quickly dissolve and destroy the morphology of the fibers of the fibrous membrane, the solvent may be mixed with one or more non-solvents to provide a diluted solvent formulation that is capable of providing controlled swelling and welding of the fibers together, without destroying the morphology of these fibers. In some embodiments, the solvent is selected from one or more of acetone, methyl ethyl ketone, dimethylacetamide, ethyl acetate, methyl acetate, N-methyl pyrrolidone, propylene carbonate, lactate esters, diethyl ether, dichloromethane, tetrahydrofuran, ethanol and methanol. In some embodiments, the solvent is acetone in concentrated form, such as formulations approaching 100% pure acetone (NEAT). In some embodiments, the acetone is diluted with a polar non-solvent, such as isopropanol, to provide a formulation comprising 20-80% acetone. In certain embodiments, the acetone has been diluted to a concentration of 50% or 25%.

Methods by which the solvent is applied to the fibrous membrane are not particularly limited, so long as the solvent is applied relatively uniformly to at least one side of the fibrous membrane. In some embodiments, the solvent may be applied to the fibrous membrane before the membrane is contacted with the sacrificial substrate. In some embodiments, the fibrous membrane is saturated with acetone. Saturation of the fibrous membrane with solvent may be carried out through soaking, dipping or spraying, in combination with a duration of time that permits saturation. In some embodiments, it is not desirable to saturate the fibrous membrane with a solvent. In these circumstances, the solvent may be applied to a surface of the fibrous membrane in limited amounts, such as by spraying. In some embodiments, 0.5-10 mL of solvent is applied per 100 cm² of fibrous membrane. In some embodiments, 0.5-1.5 mL, 1.0-2.5 mL, 2.0-4.0 mL, 3.0-4.5 mL, 4.0-5.5 mL, 5.0-6.5 mL, 6.0-7.5 mL, 7.0-8.5, or 8.0-9.5 mL of solvent is applied per 100 cm² of fibrous membrane.

In some embodiments, the solvent welding is accompanied by the application of compression to further facilitate induction of morphological change in the fibrous membrane when applied over a sacrificial substrate and/or to enhance the weld between the fibers of the fibrous membrane. The particular pressure utilized in the compression step to facilitate morphological change of the membrane and welding of the fibers together can be any pressure disclosed herein and is not particularly limited, so long as the pressure does not result in collapse of the substrate to which the fibrous membrane is applied. In some embodiments, the compression is utilized in combination with solvent welding, and includes a compression force of at least 0.1-25 newtons, generated by compressing opposite release substrates that sandwich the fibrous membrane and sacrificial substrate to which the fibrous membrane is applied.

In some embodiments, application of compression during a post-fabrication solvent welding step reduces the time required to obtain a strong weld between the fibers. The particular time required to impart a strong weld under compression is not particularly limited and includes duration of times as disclosed herein. In some embodiments, the fibrous membrane is exposed to pressure and/or solvent for at least 30 seconds, 45 seconds, 60 seconds, 75 seconds, 90 seconds, 105 seconds, 120 seconds, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes or more.

In some embodiments, the post-fabrication treatment includes physical or chemical cross-linking of the fibers. In some embodiments, the crosslinking is achieved by exposure to ultraviolet light, gamma-radiation or plasma, by inclusion of a one or more crosslinking agents, or combinations thereof. The crosslinking agents that can be used to facilitate crosslinking of the fibers are not particularly limited, but include those which induce the formation of a covalent bond between compounds found in adjacent fibers. In some instances, the crosslinking agents include glyoxal, isocynates, glutaraldehyde, formaldehyde, carbodiimides, epoxides, citric acid, tannin, ferulic acids, glyceraldehyde, genepin or transglutaminase.

In some embodiments the post-fabrication treatment alters the morphology and/or enhances the mechanical properties of the fibrous membrane. The enhanced mechanical properties may include, but are not limited to one or more of enhanced tensile strength, enhanced tensile modulus, enhanced abrasion resistance, enhanced thermal stability, enhanced elongation at break, enhanced hardness, and enhanced crystallinity. Changes in morphology include, but are not limited to, one or more of increase in fiber diameter, decrease in fiber diameter, increase in porosity, decrease in porosity, increase in pore tortuosity, and decrease in pore tortuosity.

In some embodiments, once the sacrificial material is completely surrounded by fibrous membrane, the apparatus can be further modified with additional channel networks or other tissue material that may or may not contain cells. Next the vascular network is formed by removal of the sacrificial material, creating a device consisting of (1) a hollow lumen that may or may not contain hierarchical channel network(s) completely surrounded by a (2) fibrous basement membrane separating the vascular network from (3) adjacent bulk tissue or channel networks.

The fibrous membrane comprises fibers, preferably nanofibers, randomly aligned or with specific orientation fabricated in a non-woven fashion, preferably by electrospinning.

In some embodiments, the fibers comprise a single material or blend of materials, such as from gelatin, gelatin composites, collagen, fibrin, chitosan, nitrocellulose, polylactic acid, polycaprolactone, polyethylene glycol, polyethylene glycol diacrylate or other biopolymers, polymers, or decellularized tissue or extra-cellular matrix that has been liquefied or homogenized. In a preferred embodiment, the fibers comprise a combination of gelatin and polycaprolactone.

When fabricated into nanofibers, these materials can exhibit highly desirable mechanical properties and are strong enough to form a thin membrane capable of withstanding physiologic pressures. The composition of the nanofiber membrane also allows for a highly controlled porosity that can be used for filtration, diffusion, or other such transport of specific molecules, while excluding others based on size. This allows for module of a variety of functions, including immune sensing and response.

In some embodiments, the fibrous membrane is created by electrospinning fibers directly onto a sacrificial material or template that forms a vascular network. This can be accomplished in a uniform manner by altering the electric field of the system including tailoring the voltage, current, and polarity of the nozzles, collectors, sacrificial material, and auxiliary guides. A similar result can also be achieved by dip coating, spraying, or otherwise adhering a layer of fibers suspended in solution/solvent onto a sacrificial material or template that forms a vascular network.

Additional examples of this invention include devices where the sacrificial material is PVA, BVOH, Poloxomers P407, sucrose, or other water-soluble sacrificial material. In particular, water-soluble sacrificial materials include water soluble polyurethanes. Especially envisioned are those water soluble polyurethanes which are non-toxic to human kidney fibroblasts and which can quickly dissolve in water at room temperature or 37° C. under neutral pH without obvious swelling are envisioned.

Additional examples of this invention include devices where the tissue material is gelatin methacryol, fibrin, collagen, methylcellulose, homogenized extracellular matrix, or others.

Additional examples of this invention include devices where multiple constructs or components are assembled and functionally joined together by deposition of additional membrane material, ultrasonic welding, solvent bonding, adhesives, or other techniques, so that they form a continuous basement membrane throughout all constructs or components.

Additional examples of the invention include membranes as described in the previous examples, containing hydrogels, polymers, and compounds of materials which have been modified via addition of enhancing agents or compounds in order to yield tunable mechanical and biological properties.

Additional examples of the invention include membranes as described in the previous examples where the membrane is fabricated in a multi-step process in order to create a membrane of mixed composition or architecture.

Additional examples of the invention include membranes as described in the previous examples, containing hydrogels and polymers with the encapsulation or addition of biological factors to promote cell and tissue growth.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

As used herein “A and/or B”, where A and B are different claim terms, generally means at least one of A, B, or both A and B. For example, one sequence which is complementary to and/or hybridizes to another sequence includes (i) one sequence which is complementary to the other sequence even though the one sequence may not necessarily hybridize to the other sequence under all conditions, (ii) one sequence which hybridizes to the other sequence even if the one sequence is not perfectly complementary to the other sequence, and (iii) sequences which are both complementary to and hybridize to the other sequence.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### EXAMPLES

### Example 1

### Fibrous Basement Membrane Manufacture

A sacrificial material was used to fabricate a vascular structure by extrusion 3D printing with the desired hierarchical branching vascular channel architecture, made of a water soluble polyurethane.

A mixture of gelatin and polycaprolactone (PCL) (1:1 by weight) were then dissolved in a solvent containing 8 parts acetic acid and 1 part formic acid, to achieve a final concentration of 9% wt gelatin and 9% wt PCL.

The vascular structure made from sacrificial material was then mounted into an electrospinning machine on a motorized stage. The gelatin and PCL solution was loaded into the system and fibers were generated through standard electrospinning conditions, with a voltage range from 8000-20000 volts, current from 0.1-50 mA. Fluid flow rates depend on nozzle count and configuration. Scanning across, rotation of, and reorientation of the vascular channel sacrificial material in conjunction with the electrospinning configuration was used to create a full and continuous membrane coating. Additional manipulation of the electric field by using oppositely charged nozzles facing each other was used to allow for complete circumferential coating of the sacrificial material, in a process referred to as “bipolar electrospinning.” Once the sacrificial material was fully coated, the construct was removed from the electrospinning system. The complete construct had an exposed outer membrane surface and an inner vascular surface that is in contact with the sacrificial material.

Additional sacrificial material was then deposited onto the outer membrane surface of the membrane material in order to create epithelial channel networks or spaces.

A secondary solution of 17% wt. gelatin in phosphate buffered saline was then prepared.

This secondary solution was deposited into a mold, the vascular pattern and associated membrane were added onto the solution surface, and a second volume of solution was added to the mold so that the vascular pattern and membrane material were fully encased in the tissue solution.

The tissue solution was then crosslinked by addition of transglutaminase (10U/g gelatin) and allowed to cure at 4° C. for 24 hours, forming a single tissue construct containing a hierarchical vascular network surrounded by an epithelial channel network, with the fiber basement membrane fully delineating the interface between the two.

### Example 2

The experiment was to generate a tube structure of known size, with and without support, based on the following steps:

3 mm ID mandrel placed in mold, with or without fiber support tube in place.

The mold is then filled with 12.5% wt. gelatin solution and allowed to solidify at 4° C. for 1 hour.

The mandrel is then removed from the mold with the gelatin structure.

The gelatin structure is then removed from mandrel and placed in solution with 10U of transglutaminase for 1 hour at room temperature.

The gelatin structure is mounted on barbed wire connectors, held in place with silk sutures.

One end of the gelatin tube is capped and one is connected to a pressure sensor and syringe.

The syringe barrel is compressed at a rate equal to 50 ml/min until a rupture occurs with data collected by pressure sensor.

TABLE 1

| Design | Pressure | Average | STDV |
|---|---|---|---|
| Fiber | 475 | 495 | 106.419 |
| Fiber | 610 | | |
| Fiber | 400 | | |
| No Fiber | 38 | 42.66667 | 8.082904 |
| No Fiber | 52 | | |
| No Fiber | 38 | | |

1. An apparatus comprising one or more channels defining a luminal space, wherein the luminal space is defined by an inner wall of fibrous basement membrane material comprising electrospun fibers.

2. The apparatus of claim 1, wherein the one or more channels form a vascular channel network.

3. The apparatus of claim 2, wherein the vascular channel network has a first end configured to be in fluid communication with a fluid supply and a second end configured to connect in fluid communication with a fluid outlet.

4. The apparatus of claim 1, further comprising a scaffold material contacting an outer wall of the fibrous basement membrane material.

5. The apparatus of claim 4, wherein the scaffold material comprises one or more additional vascular channel networks that, optionally, are defined by an inner wall of fibrous basement membrane material.

6. The apparatus of claim 4, wherein the scaffold material comprises cells or bulk tissue.

7. The apparatus of claim 5, wherein the scaffold material has insufficient mechanical strength to define the luminal space in the absence of the fibrous basement membrane material support.

8. The apparatus of claim 1, wherein the inner wall of fibrous basement membrane material inhibits or prevents an immunological reaction by a subject to cells and other substances in the luminal space when implanted in the subject.

9. The apparatus of claim 1, wherein the fibrous basement membrane material comprises pores of sufficient size to enable diffusion of one or more biologically relevant molecules.

10. The apparatus of claim 1, wherein the one or more channels are capable of withstanding at least 60 mmHg internal pressure.

11. (canceled)

12. The apparatus of claim 1, wherein the fibrous basement membrane material comprises one or more of gelatin, gelatin composites, collagen, fibrin, chitosan, nitrocellulose, polylactic acid, polycaprolactone, polyethylene glycol, polyethylene glycol diacrylate or other biopolymers, polymers or decellularized tissue or extra-cellular matrix that have been liquefied or homogenized.

13. (canceled)

14. The apparatus of claim 1, wherein the electrospun fibers comprise a first component selected from the group consisting of polycaprolactone, polyethylene glycol, and polyethylene glycol diacrylate, and a second component selected from the group consisting of gelatin, collagen and fibrin, and wherein the fibrous basement membrane has a thickness of 0.5-30 micrometers.

15. The apparatus of claim 1, wherein the fibrous basement membrane material has been subjected to one or more post-fabrication treatments selected from compression, annealing, chemical crosslinking, stretching, drawing, heat treatment, and solvent welding, whereby the treated fibrous basement membrane material is imparted enhanced mechanical properties compared to a fibrous basement membrane material not receiving one or more of these post-fabrication treatments.

16. The apparatus of claim 15, wherein the enhanced mechanical properties are selected from the group consisting of enhanced tensile strength, enhanced tensile modulus, enhanced abrasion resistance, enhanced thermal stability, enhanced elongation at break, enhanced hardness, enhanced crystallinity and combinations thereof.

17. The apparatus of claim 15, wherein the post-fabrication treatment comprises solvent welding.

18. The apparatus of claim 17, wherein the solvent welding is conducted in the presence of pressure imparted by opposing support substrates.

19. The apparatus of claim 15, wherein the post-fabrication treatment comprises heat treatment in combination with pressure imparted by opposing support substrates.

20. The apparatus of claim 3, wherein the apparatus is implanted in a subject or is extracorporeal to a subject.

21. (canceled)

22. The apparatus of claim 1, wherein the luminal space comprises one or more cells selected from endothelial cells, epithelial cells, mesenchymal cells, induced pluripotent stem cell derived cells, endocrine cells, and stromal cells.

23. The apparatus of claim 1, wherein the apparatus is configured to function as an artificial kidney, pancreas, lung, heart muscle, liver, spleen, small bowel, large bowel, neural tissue, skeletal muscle, composite tissue, fat tissue, bone tissue, or skin.
